(19) United States
(12) Patent Application Publication
(10) Pub. No.: US 2013/0261640 A1
(43) Pub. Date: Oct. 3, 2013

(54) SURGICAL ROBOT SYSTEM AND METHOD OF CONTROLLING THE SAME

(71) Applicant: SAMSUNG ELECTRONICS CO., LTD., Suwon (KR)

(72) Inventors: Hyung-joo KIM, Seongnam (KR); Yeon-ho KIM, Hwaseong (KR); Hyun-do CHOI, Yongin (KR)

(73) Assignee: SAMSUNG ELECTRONICS CO., LTD., Suwon (KR)

(21) Appl. No.: 13/851,586

(22) Filed: Mar. 27, 2013

(30) Foreign Application Priority Data
Mar. 28, 2012 (KR) 10-2012-0031826

(51) Int. Cl. A61B 19/00

(52) U.S. Cl. CPC A61B 19/2203
USPC 606/130

(57) ABSTRACT

A robot system including a surgical robot inserted into a subject may include: a photographing device that is disposed on an end portion of the surgical robot, is inserted into a body part of the subject, which is to be treated, and captures a real-time image with a resolution at which the body part to be treated may be observed in units of cells; a control device that receives a control signal for controlling the surgical robot by referring to the real-time image received from the photographing device; and an energy generating device that is disposed adjacent to the photographing device, and transmits energy to a region corresponding to the body part to be treated which is being photographed by the photographing device according to the control signal.

### CROSS-REFERENCE TO RELATED APPLICATIONS

This application claims the priority benefit of Korean Patent Application No. 10-2012-0031826, filed on Mar. 28, 2012, in the Korean Intellectual Property Office, the disclosure of which is incorporated herein in its entirety by reference.

### BACKGROUND

1. Field

Example embodiments of the present disclosure relate to robot systems including surgical robots and methods of controlling the robot systems.

2. Description of the Related Art

In the medical field, a method of performing a surgical operation may include the use of a robot. The robot may be a surgical robot that is used instead of a surgeon to treat a patient's body part that requires treatment. While a robot makes decisions and performs a surgical operation in a few cases, a robot usually is used to assist a surgeon in most cases. In other words, a surgeon decides a patient's body part to be treated and a treatment method and a robot performs a surgical operation, such as, incision or injection, according to the surgeon's decision.

When a surgical operation is performed with the use of a robot, high precision and minimally invasive surgery is ensured compared to that when a surgical operation is directly performed by a surgeon. Accordingly, many studies have recently been made on a method of performing a surgical operation by using a robot. As such, there is a need for an improved surgical robot system and method for controlling the surgical robot system.

### SUMMARY

Additional aspects and/or advantages will be set forth in part in the description which follows and, in part, will be apparent from the description, or may be learned by practice of the disclosure.

Provided are surgical robot systems which may determine and treat a body part to be treated in units of cells. Provided are methods of controlling the surgical robot systems. Provided are computer-readable recording media having embodied thereon programs for executing the methods. Additional aspects will be set forth in part in the description which follows and, in part, will be apparent from the description, or may be learned by practice of the presented embodiments.

According to an aspect of the present disclosure, a robot system including a surgical robot inserted into a subject includes: a photographing device that is disposed on an end portion of the surgical robot, is inserted into a body part of the subject, which is to be treated, and captures a real-time image with a resolution at which the body part to be treated may be observed in units of cells; a control device that receives a control signal for controlling the surgical robot by referring to the real-time image received from the photographing device; and an energy generating device that is disposed adjacent to the photographing device, and transmits energy to a region corresponding to the body part to be treated which is being photographed by the photographing device according to the control signal.

According to another aspect of the present disclosure, a method of controlling a robot system including a surgical robot inserted into a subject includes: inserting the surgical robot into a body part of the subject, which is to be treated; receiving a control signal for controlling the surgical robot by referring to a real-time image with a resolution at which the body part to be treated may be observed in units of cells by using a photographing device disposed on an end portion of the surgical robot; and controlling an energy generating device that transmits energy to a region corresponding to the body part to be treated according to the control signal, by referring to the real-time image.

According to another aspect of the present disclosure, a method of controlling a robot system is provided, including: inserting a surgical robot into a body part of a subject according to a diagnostic image captured by an imaging device; moving the surgical robot to a region of the body part to be treated based on an image captured by a photographing device; and transmitting energy to the region of the body part to be treated.

According to another aspect of the present disclosure, a robot system is provided, including: an imaging device to obtain a diagnostic image; a control device to control a surgical robot; and a surgical robot to be inserted into a subject's body part to be treated, wherein the surgical robot includes a photographing device to capture an image of the body part to be treated and an energy generating device to transmit energy in a direction towards a region of the body part to be treated.

According to another aspect of the present disclosure, a computer-readable recording medium has embodied thereon a program for executing the method.

### BRIEF DESCRIPTION OF THE DRAWINGS

These and/or other aspects will become apparent and more readily appreciated from the following description of the embodiments, taken in conjunction with the accompanying drawings of which:

FIG. 1 is a perspective view illustrating a robot system, according to an example embodiment of the present disclosure;

FIG. 2 is a block diagram illustrating the robot system of FIG. 1, according to an example embodiment of the present disclosure;

FIG. 3 is a block diagram illustrating the robot system of FIG. 1, according to another example embodiment of the present disclosure;

FIG. 4A is a view illustrating the robot of FIG. 1, according to an example embodiment of the present disclosure;

FIG. 4B is a perspective view illustrating the robot of FIG. 4A;

FIG. 5 is a perspective view illustrating the robot of FIG. 1, according to another example embodiment of the present disclosure;

FIG. 6 is a perspective view illustrating the robot of FIG. 1, according to another example embodiment of the present disclosure;

FIG. 7 is a perspective view illustrating the robot of FIG. 1, according to another example embodiment of the present disclosure; and

FIG. 8 is a flowchart illustrating a method of controlling the robot system, according to an example embodiment of the present disclosure.

### DETAILED DESCRIPTION

As used herein, the term “and/or” includes any and all combinations of one or more of the associated listed items. Expressions such as “at least one of,” when preceding a list of elements, modify the entire list of elements and do not modify the individual elements of the list.

The present disclosure will now be described more fully with reference to the accompanying drawings, in which exemplary embodiments of the disclosure are shown.

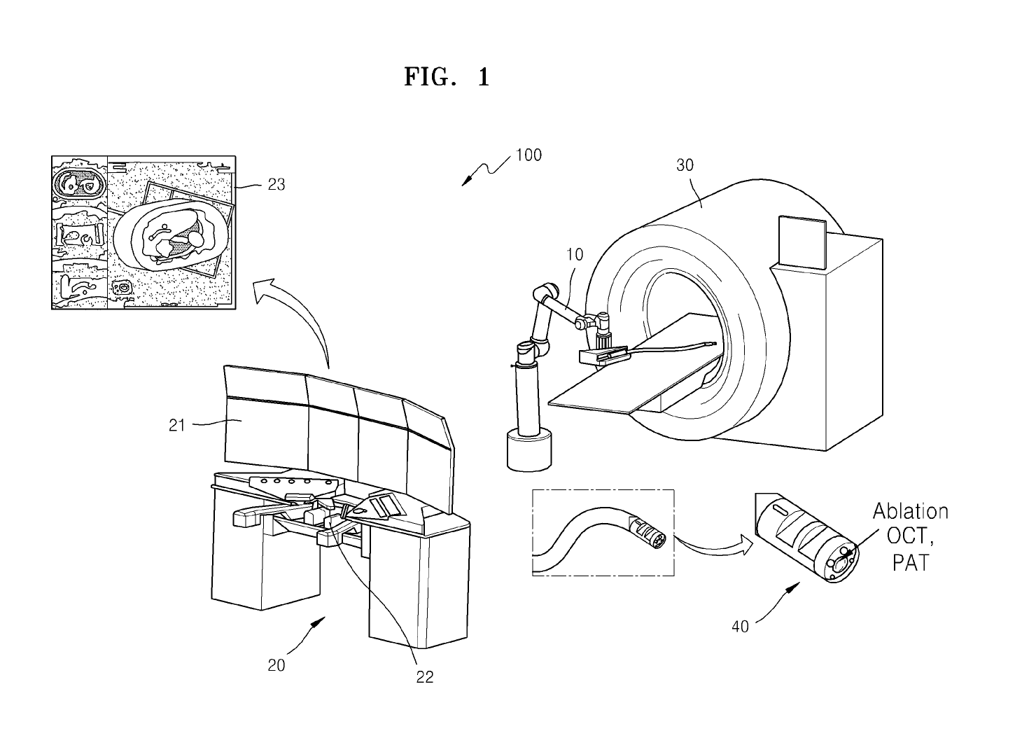

FIG. 1 is a perspective view illustrating a robot system 100, according to an example embodiment of the present disclosure. Referring to FIG. 1, the robot system 100 may include a robot 10, a control device 20, and an imaging device 30.

The robot system 100 may insert the robot 10 into a subject's body part to be treated and captures a real-time image with a resolution at which the body part to be treated may be observed in units of cells. Further, the robot system 100 may determine a position of a tumor cell by referring to the real-time image and transmit energy for removing the tumor cell to the body part to be treated.

For example, the robot system 100 may move to reach a tumor cell of a subject's target organ with the help of the imaging device 30, photograph the tumor cell in the target organ in real time, and transmit energy to a nanomaterial attached to the tumor cell, without affecting cells other than the tumor cell, so as to remove the tumor cell.

In order to insert the robot 10 into a subject's body part to be treated, the robot system 100 may insert the robot 10 into a region adjacent to the body part to be treated by using a diagnostic image generated by the imaging device 30. Alternatively, the robot system 100 may insert the robot 10 according to a position of the body part to be treated and move the robot 10 to the region adjacent to the body part to be treated.

The imaging device 30 is a device for generating a diagnostic image indicating information about the inside of a subject's body. Depending on embodiments, the diagnostic image may be generated, such that the diagnostic image includes a region of the body part of the subject that is to be treated. The imaging device 30 may output the diagnostic image to the control device 20, and the control device 20 may generate a three-dimensional (3D) coordinate system indicating a position of the robot 10 and a position of the subject by referring to the diagnostic image. In detail, the control device 20 may set an arbitrary point as the center of the 3D coordinate system, and generate a 3D coordinate system indicating positions of the robot 10, the imaging device 30, and the subject based on the center of the 3D coordinate system. For example, the control device 20 may set a specific point of the robot 10 as a center, and generate a 3D coordinate system indicating positions of the robot 10, the imaging device 30, and the subject based on the specific point of the robot 10. In this case, a diagnostic image may be a two-dimensional (2D) or 3D image generated when the imaging device 30 photographs the subject.

The robot system 100 may insert the robot 10 into the body part to be treated by referring to the 3D coordinate system generated by the control device 20. In detail, the robot system 100 receives coordinates to which the robot 10 is to move for the purpose of treatment through a control unit 22 of the control device 20 and moves the robot 10 to the coordinates.

The robot 10 is inserted into the subject's body part, captures a real-time image such that the body part to be treated may be observed in units of cells (in other words, such that the cells of the subject may be observed and are visible), and transmits energy to the body part to be treated after or during photographing.

The robot 10 is inserted into the subject's body part by an operator as desired. The robot 10 moves to coordinates input from the control device 20 or moves along a preset path that may be set by the operator. For example, when current coordinates of the robot 10 are (2, 3, 4) and coordinates input from the control device 20 are (5, 6, 7), the control device 20 moves the robot 10 such that the robot 10 is located on the coordinates (5, 6, 7). In this case, the current coordinates of the robot 10 may indicate coordinates of an end portion 40 of the robot 10. Also, when a movement direction (e.g., upward, downward, leftward, or rightward) is input from the control device 20, the robot 10 moves in the indicated movement direction.

The robot 10 captures a real-time image with a resolution at which the body part to be treated may be observed in units of cells and outputs the real-time image. Further, the real-time image may be obtained, such that the real-time image includes an image of the part of the subject's body to be treated. The robot 10 including a photographing device for observing cells of the subject obtains a real-time image captured by the photographing device and outputs the real-time image to the control device 20.

In addition, the robot 10 transmits energy to a region having a cell size corresponding to the body part to be treated which is being or has been photographed. The robot 10 may recognize a cell based on the resolution of the real-time image having units of cells, such that the cells may be observed. Thus, the robot 10 may transmit energy to cells to be treated. In order to transmit the energy, the robot 10 may include an energy generating device for transmitting energy to a region having the corresponding cell size. The energy generating device may transmit energy to a cell to be removed. The robot 10 receives a control signal for transmitting energy from the control device 20, and transmits energy to the region corresponding to the body part to be treated by using the energy generating device.

The control device 20 receives a control signal for controlling the robot 10 by referring to the real-time image received from the robot 10. The control device 20 displays the real-time image received from the robot 10, and receives from a user or operator a control signal for moving the robot 10 and generating energy to be transmitted by the energy generating device. The user or operator may be a surgeon or a medical expert, for example. The control device 20 may receive a control signal related to coordinates or movement, and may receive a control signal related to energy, such as, type, intensity, range, or transmission angle, and the like. The control device 20 controls the robot 10 according to a control signal related to movement and energy, to move the robot 10 or to enable the robot 10 to generate energy, through the energy generating device, to be transmitted to the body part to be treated.

The control device 20 may include a display device 21 and the control unit 22. The display device 21 displays a real-time image received from the robot 10. The control unit 22 receives a control signal for controlling the robot 10. In FIG. 1, an image 23 may be an image showing the inside of the subject's body which is output in real time from the display device 21, or a diagnostic image which is received from the imaging device 30. The image showing the inside of the subject's body may be a real-time image, and the diagnostic image may be a real-time image or a non-real-time image. Further, the image 23 may have a resolution, such that the cells of the part of the subject's body being photographed may be visible.

The imaging device 30 outputs a diagnostic image generated by photographing the subject to the control device 20. The diagnostic image generated by the imaging device 30 may be a 2D or 3D image, and may be used when the control device 20 moves the robot 10. In detail, if the diagnostic image is a 3D image, the diagnostic image may be used to obtain a 3D coordinate system of the subject, and the control device 20 may determine coordinates to which the robot 10 is to move based on the 3D coordinate system. Alternatively, if the diagnostic image is a 2D image, the diagnostic image may be used to obtain a 2D coordinate system.

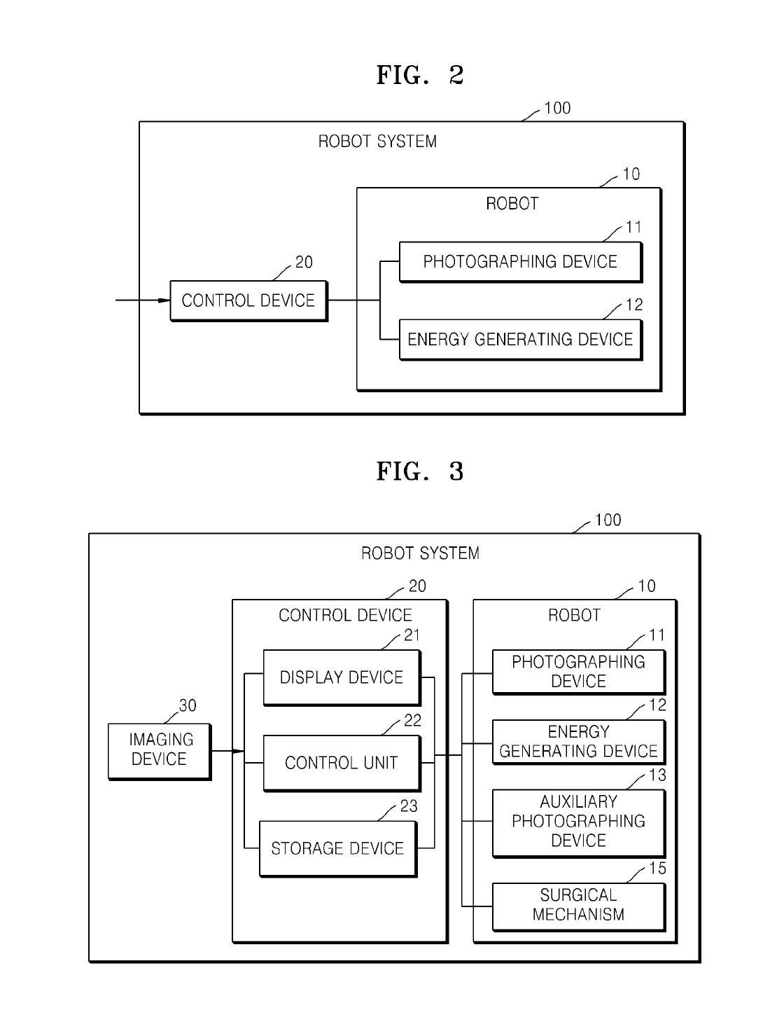

FIG. 2 is a block diagram illustrating the robot system 100 of FIG. 1, according to an example embodiment of the present disclosure. Referring to FIG. 2, the robot system 100 includes the control device 20 and the robot 10. Also, the robot 10 includes a photographing device 11 and an energy generating device 12. Since FIG. 2 illustrates a portion of the robot system 100 of FIG. 1, although omitted here, the description of the robot system 100 given in relation to FIG. 1 may apply to the robot system 100 of FIG. 2.

FIG. 2 illustrates elements of the robot system 100 related to the present embodiment. Accordingly, it will be understood by one of ordinary skill in the art that the robot system 100 may further include general-purpose elements other than the elements illustrated in FIG. 2.

The robot 10 includes the photographing device 11 and the energy generating device 12. The photographing device 11 captures a real-time image with a resolution at which a body part to be treated may be observed in units of cells. Also, the photographing device 11 may capture a real-time image with a resolution, such that the cells of the part of the subject's body being photographed are visible to the user or operator. Examples of the photographing device 11 may include a fluorescence imaging device, a high-resolution microendoscope device, an optical coherence tomography (OCT) device, a photoacoustic transducer (PAT) device, and a confocal microendoscope device. However, the present disclosure is not limited thereto, and thus, the photographing device 11 may be other devices as long as they may be used to observe the body part to be treated in units of cells.

Since the photographing device 11 captures a real-time image with a resolution at which the body part to be treated may be observed in units of cells and outputs the real-time image to the control device 20, an operator may see a tumor cell by using the real-time image which is being displayed. Also, since a process of removing the tumor cell is photographed in real time by using the photographing device 11, the operator may monitor a result after operation and determine whether any part of the tumor cell remains, for example, after the energy generating device has transmitted energy to the part of the subject's body being treated.

Moreover, the robot 10 may include an auxiliary photographing device in addition to the photographing device 11. The auxiliary photographing device may be a general camera or an endoscope, for example. If the auxiliary photographing device is included in the robot 10, the auxiliary photographing device may be disposed on a front surface of the end portion 40 of the robot 10, photograph in a travel direction of the robot 10, and output a real-time image or a non-real-time image to the control device 20.

Further, the energy generating device 12 may be disposed near the photographing device 11. For example, the energy generating device 12 may be disposed adjacent to the photographing device 11. Moreover, the energy generating device 12 may transmit energy to a region having a cell size corresponding to the body part to be treated which is being photographed by the photographing device 11. Examples of the energy generating device 12 may include a laser generating device, a light-emitting diode (LED), a radio frequency (RF) signal generating device, and a microwave signal generating device. However, the above types of energy generating devices are exemplary, and thus, the present disclosure is not limited thereto.

The energy generating device 12 transmits energy to a material which may react to the energy. For example, a nanomaterial or a molecular material which is attached to a tumor cell and reacts to specific energy is directly injected into an organ, injected through a urethra, or injected through a blood vessel. The nanomaterial or the molecular material including a component for destroying the tumor cell may be selectively attached to the tumor cell. The nanomaterial is activated by reacting to energy, and thus, the tumor cell is destroyed. Alternatively, the nanomaterial reacts to energy to release the component for removing the tumor cell, and thus, the tumor cell is removed. The nanomaterial or the molecular material is activated by receiving energy from the energy generating device 12.

Since the energy generating device 12 may transmit energy to a region having a cell size in the above-described manner, the energy generating device 12 may transmit energy to the nanomaterial attached to the tumor cell by referring to a real-time image received from the photographing device 11. Accordingly, since the nonmaterial is not attached to a healthy cell, energy is not transmitted to a healthy cell, but only to the tumor cell, and thus, only the tumor cell may be removed without causing damage to healthy cells of the subject's body.

In the case of a drug delivery system, since a nanomaterial is applied to only a specific cell, a higher density of medicine may be locally used and the effect on the entire body may be minimized. In other words, if a component included in a nanomaterial is activated in a healthy cell other than a tumor cell, the healthy cell may be destroyed. However, since the energy generating device 12 may transmit energy only to a region where a tumor cell exists, a nanomaterial is not activated in a healthy cell, thereby avoiding damaging healthy cells. Accordingly, since the density of a component included in the nanomaterial may be increased, the tumor cell may be efficiently removed, and the effect of the component included in the nanomaterial on the healthy cell may be minimized.

In addition, an energy transmission range of the energy generating device 12 may vary according to an energy transmission depth. For example, when precise ablation is not needed, an energy source having a wider energy transmission range is used. However, when a tumor is removed, since precise ablation is needed so as not to damage adjacent tissues, an energy source having a precise energy transmission range or a narrower transmission range is used, thereby avoiding damaging healthy cells.

The control device 20 controls the photographing device 11 and the energy generating device 12 included in the robot 10. The control device 20 receives a control signal for controlling the photographing device 11 and the energy generating device 12 by referring to a real-time image received from the photographing device 11. For example, the control device 20 may be an electronic device including a display unit and a control unit, such as a computer. The display unit may be a monitor for displaying a real-time or non-real-time image received from the photographing device 11, and the control unit may be a keyboard, a mouse, or a joystick that receives a number or a direction from a user. It will be understood by one of ordinary skill in the art that the display unit and the control unit are exemplary, and thus, the present disclosure is not limited thereto.

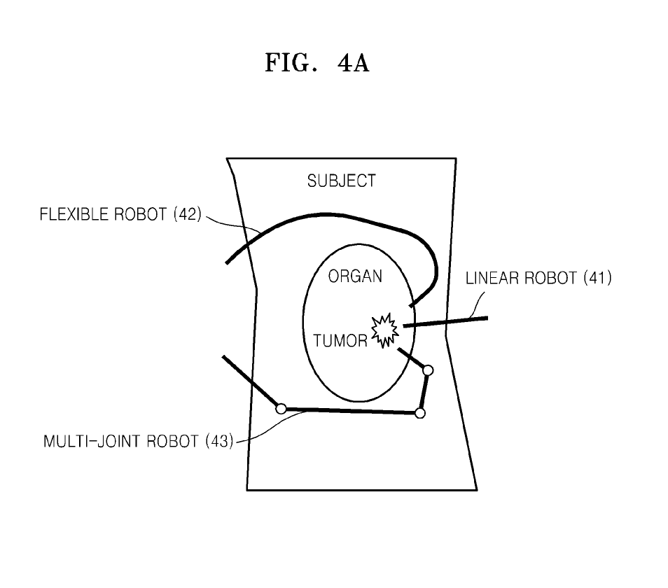

FIG. 3 is a block diagram illustrating the robot system 100 of FIG. 1, according to another example embodiment of the present disclosure. Since FIG. 3 illustrates a portion of the robot system 100 of FIG. 1, although omitted here, the description of the robot system 100 given in relation to FIG. 1 also applies to the robot system 100 of FIG. 3. In addition, since the robot system 100 of FIG. 3 includes additional elements than the robot system 100 of FIG. 2, the description of the robot system 100 given in relation to FIG. 2 also applies to the robot system 100 of FIG. 3. Referring to FIG. 3, the robot system 100 includes the imaging device 30, the control device 20, and the robot 10.

The imaging device 30 generates an image indicting information about the inside of a subject's body, and outputs the image to the control device 20. For example, the imaging device 30 may be a medical device that may show the inside of a subject's body, such as an ultrasound imaging device, a computed tomography (CT) device, or a magnetic resonance imaging (MRI) device, however, these devices are exemplary, and thus, the present disclosure is not limited thereto.

In particular, if the imaging device 30 is an MRI device, for example, the imaging device 30 enables the subject to lay in a tube where a magnetic field is generated, generates a high frequency signal to resonate hydrogen atomic nuclei in the subject, and generates a diagnostic image by using a difference between signals output from tissues.

If the imaging device 30 is an ultrasound imaging device, for example, the imaging device 30 transmits a source signal generated from a probe mounted on the imaging device 30 to an observation region in the subject to be diagnosed. Further, the imaging device 30 may generate image data of volume images indicating the observation region by using a reaction signal generated by the source signal. The source signal may be any of various signals, such as, an ultrasound signal and an X-ray signal, however, the present disclosure is not limited thereto.

In this regard, examples of a diagnostic images generated by the imaging device 30 may include various medical images, such as, an ultrasound image, an X-ray image, and an MRI image, and the like. In other words, the diagnostic image should not be limited to one type of image such as an MRI image or a CT image. Further, the diagnostic image may be a real-time image or a non-real-time image, depending on embodiments.

The diagnostic image may be a 2D image or a 3D image, depending on embodiments. In other words, the diagnostic image may be a 2D image in which a shape of a section or a predetermined observation region in the subject's body is represented by an X-axis and a Y-axis, or a 3D image in which a shape is represented by an X-axis, a Y-axis, and a Z-axis.

The control device 20 may include the display device 21, the control unit 22, and a storage device 23. The display device 21 displays a diagnostic image received from the imaging device 30 or a real-time image received from the photographing device 11. For example, the display device 21 may be a liquid crystal display (LCD) or a plasma display panel (PDP), however, the present embodiment is not limited thereto.

The control unit 22 may receive a control signal for controlling the robot 10, move the photographing device 11 to a body part to be treated, and control the energy generating device 12 to transmit energy. For example, the control unit 22 may be an electronic device, such as, a mouse, a keyboard, or a joystick, however, the present disclosure is not limited thereto.

The control unit 22 may receive a control signal that is generated from a medical expert or operator controlling, and thereby move the robot 10 according to the generated control signal. For example, the control unit 22 may receive coordinates to which the robot 10 is to move, and move the robot 10 to the coordinates. In addition, the control unit 22 may receive a movement direction of the robot 10, and move the robot 10 in the movement direction.

Further, the control unit 22 may receive a control signal for transmitting energy, and control the energy generating device 12 to transmit energy, based on the control signal. For example, the control signal may be related to at least one of a type, an intensity, a range, and a transmission angle of energy generated by the energy generating device 12, however, the present disclosure is not limited thereto. The control unit 22 may determine, for example, the type, the intensity, the range, and the transmission angle of the energy generated by the energy generating device 12 according to the control signal input to the control unit 22.

The control unit 22 may control operations of the energy generating device 12 and the photographing device 11 based on the generated control signal. For example, the control unit 22 may control the energy generating device 12 and the photographing device 11 to rotate or control the robot 10 to be carried in and out through an opening.

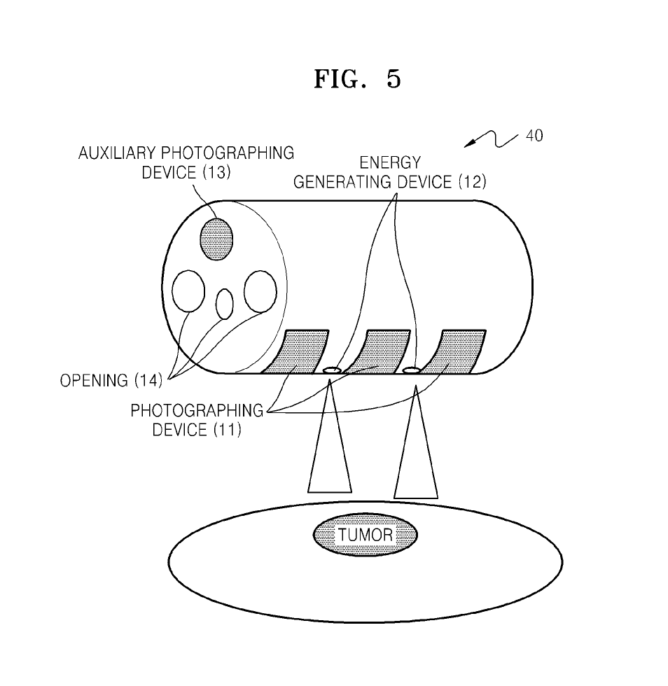

Also, depending on embodiments, the control unit 22 may set an energy transmission direction of the energy generating device 12, based on a direction in which the photographing 11 performs photographing. For example, the control unit 22 may control an operation of the energy generating device 12, such that energy is transmitted in a direction in which the photographing device 11 performs photographing even without receiving an additional control signal for the energy generating device 12. Operations of the energy generating device 12 and the photographing device 11 will be explained in detail with reference to FIGS. 5 through 7.

The storage device 23 may store an image received from the imaging device 30, the photographing device 11, or an auxiliary photographing device 13, and the like, depending on embodiments. Examples of the storage device 23 may include a hard disc drive, a read-only memory (ROM), a random access memory (RAM), a flash memory, and a memory card, however, the present disclosure is not limited thereto.

The robot 10 may include the photographing device 11, the energy generating device 12, the auxiliary photographing device 13, and a surgical mechanism 15. As an example, the photographing device 11 may be disposed on the end portion 40 of the robot 10 (refer to FIG. 1), may be inserted into a region adjacent to a subject's body part to be treated by referring to a diagnostic image indicating information about the inside of the subject's body, and may capture a real-time image with a resolution at which the body part to be treated may be observed in units of cells. Depending on embodiments, the photographing device 11 may be provided on a side surface or a front surface of the end portion 40 of the robot 10, and may be carried in and out through an opening formed in the front surface of the end portion 40 of the robot 10. Also, the photographing device 11 may rotate.

Further, for example, the photographing device 11 is inserted into a region adjacent to the body part to be treated, captures a real-time image by photographing the inside of the subject's body, and outputs the real-time image to the display device 21. Since the photographing device 11 may be located on the end portion 40 of the robot 10, when the robot 10 receives a control signal related to movement from the control unit 22 and moves according to the control signal, the photographing device 11 may move along with the robot 10. Accordingly, when the control unit 22 moves the robot 10 to the body part to be treated, the photographing device 11 may photograph the body part to be treated.

The photographing device 11 outputs to the display device 21 a real-time image which is captured during or after being moved to the body part to be treated. When the robot 10 is inserted into a region adjacent to the body part to be treated, since the photographing device 11 outputs the real-time image obtained by photographing the inside of the subject's body to the display device 21, a medical expert or operator may determine a position of the robot 20 by using the real-time image. In other words, in order for the medical expert to move the robot 10 to an exact position of the body part to be treated, the real-time image indicating the inside of the subject's body may be provided to the medical expert.

Since the photographing device 11 moves to the body part to be treated and outputs to the display device 21 a real-time image with a resolution at which the body part to be treated may be observed in units of cells (i.e., the cells are visible), the medical expert may identify a tumor cell by using the real-time image displayed on the display device 21.

Also, as another example, the robot 10 may photograph the inside of the subject's body by additionally using the auxiliary photographing device 13. The auxiliary photographing device 13 may be provided on the front surface of the end portion 40 of the robot 10, however, the present disclosure is not limited thereto. Examples of the auxiliary photographing device 13 may include a general endoscope, a high-resolution microendoscope, and a charge-coupled device (CCD) camera, however, the present disclosure is not limited thereto.

The surgical mechanism 15 is used to make an incision, stop bleeding, or inject medicine. For example, the surgical mechanism 15 may include a probe for injecting medicine or a surgical tool, such as, a laser for making an incision or stopping bleeding, however, the present disclosure is not limited thereto. Since the surgical mechanism 15 may directly inject medicine into the body part to be treated by using the probe, the possibility that the medicine is attached to the body part to be treated may be increased.

The probe may be used to inject medicine, such as, a nanomaterial or a photosensitizer, for example. The nanomaterial or the photosensitizer is a material that is activated by energy transmitted from the energy generating device 12. In detail, the nanomaterial or the photosensitizer injected from the probe may be attached to a tumor cell.

The surgical mechanism 15 may be controlled by the control unit 22. The control unit 22 may control the probe for injecting medicine, or may control the surgical tool for making an incision or stopping bleeding of the surgical mechanism 15. Also, if necessary, the control unit 22 may be directly manipulated by an operator. The control unit 22 controls an operation of the surgical mechanism 15 by referring to a real-time image output from the photographing device 11 or the auxiliary photographing device 13.

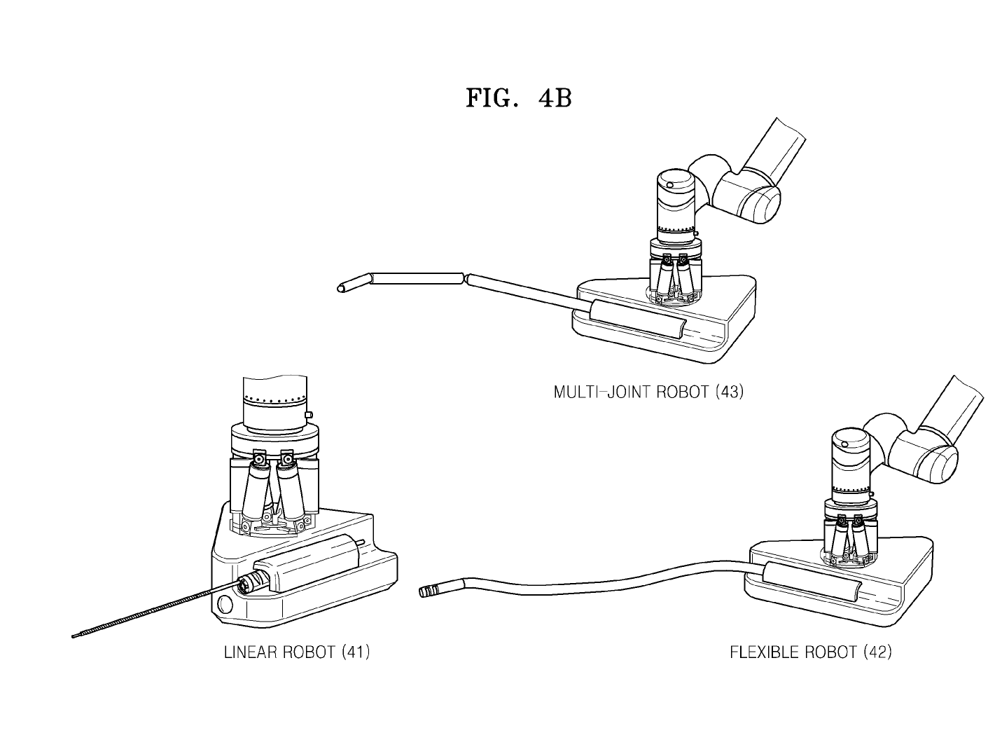

FIG. 4A is a view illustrating the robot 10 of FIG. 1, according to an example embodiment of the present disclosure. Accordingly, although omitted here, the description of the robot 10 given in relation to FIG. 1 applies to the robot system 100 of FIG. 4A. FIG. 4A illustrates different example embodiments of the robot 10 having various structures for accessing a tumor cell in a subject's body. Referring to FIG. 4A, the robot 10 may have various structures including a linear robot 41, a flexible robot 42, or a multi-joint robot 43, for example.

The linear robot 41 is a robot having a straight shape, for example, a bar shape. In other words, the linear robot 41 is not bent, and moves using a shortest path to a subject's body part to be treated. Generally, the linear robot 41 is used when a distance between a skin and the body part to be treated is short or there is no major organ between the skin and the body part to be treated. If there exists a major organ between the skin and the body part to be treated, other structures of the robot 10 may be used. However, since the linear robot 41 is straightly inserted into the subject, the linear robot 41 may accurately reach the body part to be treated.

The flexible robot 42 is a robot that is softly bent. Depending on embodiments, the flexible robot 42 may have a curved structure. For instance, when there exists a major organ between the skin and the body part to be treated, the flexible robot 42 may move to a tumor cell along a curving route so as to avoid or dodge a major organ. When there exists a major organ, since the flexible robot 42 moves by avoiding or dodging the major organ, the flexible robot 42 may not damage the major organ.

The multi-joint robot 43 is a robot including a plurality of bars which are combined using joints. In other words, since the bars are connected at joints, the bars may be bent at the joints. However, each of the bars being connected by the joints is not bent, similar to the linear robot 41. Since the multi-joint robot 43 may reach a tumor cell by being bent when there exists a major organ, like the flexible robot 42, the multi-joint robot 43 may avoid the major organ, and thereby not damage the major organ.

Since the linear robot 41 is straightly inserted into a target point, when there exists a structure between the target point and a skin, the linear robot 41 has to pass through the structure. If the structure is a major organ such as an intestine or a blood vessel, the linear robot 41 may pass through the major organ, damage the major organ, and cause serious complications. Additionally, when a tumor cell exists in several portions, the several portions of the subject have to be incised and then the linear robot 41 has to be inserted.

Accordingly, damage to a major organ may be prevented and minimally invasive surgery may be performed by using any of the linear robot 41, the flexible robot 42, and the multi-joint robot 43 according to a position of a target point and a distribution of tumor cells.

FIG. 4B is a perspective view illustrating the robot 10 of FIG. 1. FIG. 4B is a detailed perspective view illustrating the linear robot 41, the flexible robot 42, and the multi-joint robot 43 of FIG. 4A. Accordingly, the description of the linear robot 41, the flexible robot 42, and the multi-joint robot 43 given in relation to FIG. 4A applies to the linear robot 41, the flexible robot 42, and the multi-joint robot 43 of FIG. 4B.

FIG. 5 is a perspective view illustrating the robot 10 of FIG. 1, according to another example embodiment of the present disclosure. Accordingly, although omitted here, the description of the robot 10 given in relation to FIG. 1 applies to the robot 10 of FIG. 5. FIG. 5 is an enlarged view illustrating a portion of the robot 10, including the end portion 40 of the robot 10. Referring to FIG. 5, the end portion 40 of the robot 10 may include, for example, the photographing device 11, the energy generating device 12, the auxiliary photographing device 13, and an opening 14.

The photographing device 11 and the energy generating device 12 may be provided on a side surface of the end portion 40 of the robot 10. Also, the photographing device 11 and the energy generating device 12 may be arranged in a longitudinal direction of the robot 10. If a plurality of the photographing devices 11 and the energy generating devices 12 are provided, the plurality of photographing devices 11 and the plurality of energy generating devices 12 may be arranged in the longitudinal direction of the robot 10 to intersect each other. That is, as an example, the photographing device 11 and the energy generating device 12 may be provided on the side surface of the robot 10 in an alternating manner. The end portion 40 of the robot 10 is an extremity at which the robot 10 ends, and the side surface of the end portion 40 is a surface surrounding the outside of the robot 10. Also, the longitudinal direction of the robot 10 is a direction in which the robot 10 extends lengthwise from a proximal end to a distal end. Although the end portion 40 has a cylindrical shape in FIG. 5, the present embodiment is not limited thereto. The end portion 40 of the robot 10 may have any of various shapes as well as the cylindrical shape. Although the opening 14 has a circular shape in FIG. 5, the present embodiment is not limited thereto.

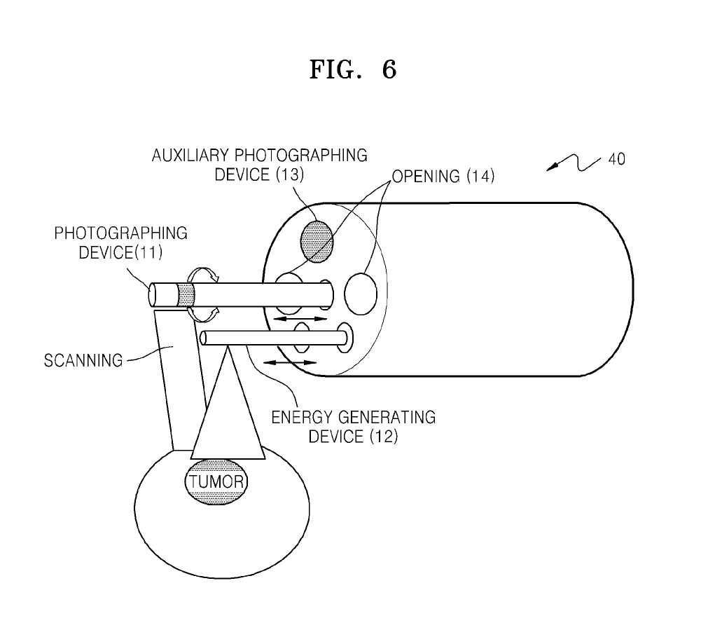

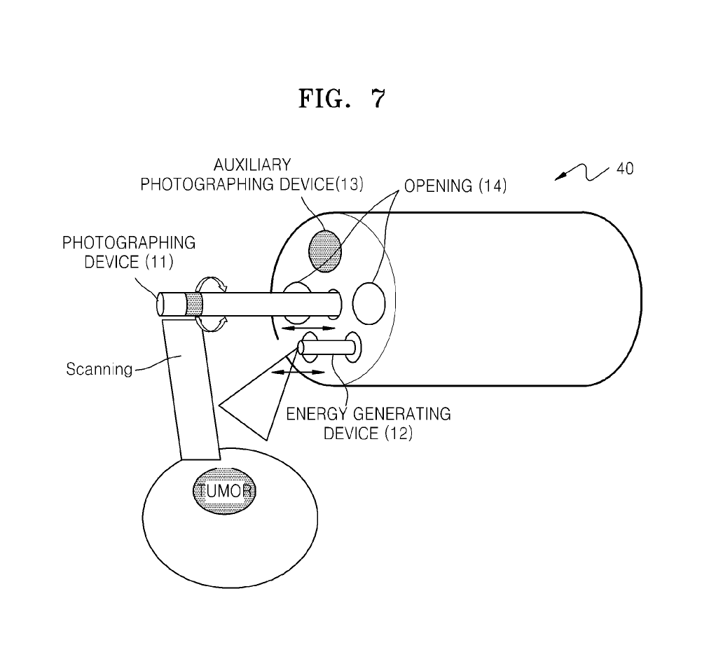

The opening 14 and the auxiliary photographing device 13 may be provided in a front surface of the end portion 40 of the robot 10. A plurality of the openings 14 may be provided, and act as paths through which various devices may be slid or be carried in and out. The photographing device 11 or the energy generating device 12 may slide through or be carried in and out through the opening 14. In other words, the photographing device 11 and the energy generating device 12 may be provided on the side surface, and an additional photographing device or the energy generating device 12 may slide through or be carried in and out through the opening 14. A structure in which the photographing device 11 and the energy generating device 12 are carried in and out through the opening 14 will be explained in detail with reference to FIGS. 6 and 7.

Also, the surgical mechanism 15 may be carried in and out through the opening 14. The surgical mechanism 15 may be a probe for injecting medicine or controlling a surgical tool for making an incision or stopping bleeding as described above.

Referring to FIG. 5, a tumor cell is disposed adjacent to the side surface of the end portion 40 of the robot 10. Accordingly, the photographing device 11 disposed on the side surface of the end portion of the robot 10 may photograph the tumor cell, and output an image to the display device 21 of the control device 20. The outputted image may be in real-time or may not be in real-time, depending on embodiments. The energy generating device 12 disposed adjacent to the photographing device 11 on the side surface of the end portion of the robot 10 may transmit energy to the tumor cell. The energy generating device 12 may set an energy transmission direction based on the direction of the photographing device 11. For example, the energy generating device 12 may set a direction to be a direction in which the photographing device 11 performs photographing under the control of the control device 20. If the photographing device 11 rotates along the side surface of the robot 10, the control device 20 may rotate the energy generating device 12 along with the photographing device 11.

Accordingly, as an example, the photographing device 11 and the energy generating device 12 may be set to automatically face the same direction, however, the present disclosure is not limited thereto. For example, an energy transmission direction of the energy generating device 12 may be manually set.

When the photographing device 11 and the energy generating device 12 are provided to face the same direction in the robot 10 constructed as described with reference to FIG. 5, it is easy for the energy generating device 12 to transmit energy to a region which the photographing device 11 photographs.

FIG. 6 is a perspective view illustrating the robot 10 of FIG. 1, according to another example embodiment of the present disclosure. Accordingly, although omitted here, the description of the robot 10 given in relation to FIG. 1 applies to the robot 10 of FIG. 6.

Referring to FIG. 6, the photographing device 11 and the energy generating device 12 may slide through or be carried in and out through the opening 14. When the photographing device 11 and the energy generating device 12 slide through or are carried in and out through the opening 14, it means that the photographing device 11 and the energy generating device 12 may move to be located inside the robot 10 or outside the robot 10 under the control of the control unit 22.

The photographing device 11 may have a cylindrical bar shape, and may rotate about a longitudinal direction of the photographing device as shown in FIG. 6. However, the photographing device 11 is not limited to a cylindrical bar shape. As only the photographing device 11 rotates, the photographing device 11 may scan surroundings of the robot 10. In other words, the photographing device 11 may rotate and output a real-time image obtained by photographing the surroundings of the display device 21. A medical expert may determine a position of a tumor cell by referring to the real-time image, and may fix the photographing device 11 to be located at the position of the tumor cell based on the control signal of the control unit 22.

The energy generating device 12 may have a cylindrical bar shape, and may rotate about a longitudinal direction of the photographing device 12, like the photographing device 11. However, the energy generating device 12 is not limited to a cylindrical bar shape. Accordingly, as the photographing device 11 rotates, the energy generating device 12 may also rotate along with the photographing device 11. Depending on embodiments, the photographing device 11 and the energy generating device 12 may not rotate together.

An energy transmission direction of the energy generating device 12 may be determined based on a direction in which the photographing device 11 performs photographing. When the energy generating device 12 and the photographing device 11 slide through or are carried in and out through the opening 14, there exists a predetermined distance between the energy generating device 12 and the photographing device 11. Accordingly, a region which the photographing device 11 photographs and a region to which the energy generating device 12 transmits energy may be matched. For example, as the photographing device 11 moves or rotates, the control unit 22 may set a direction which the energy generating device 12 faces to be a direction which the photographing device 11 faces. For example, the control unit 22 may set such that the energy generating device 12 faces the center of an image which is being captured by the photographing device 11. Alternatively, an energy transmission direction of the energy generating device 12 may be manually set.

FIG. 7 is a perspective view illustrating the robot of FIG. 1, according to another embodiment of the present disclosure. Accordingly, although omitted here, the description of the robot 10 given in relation to FIG. 1 applies to the robot 10 of FIG. 7.

Referring to FIG. 7, the photographing device 11 and the energy generating device 12 may slide through or be carried in and out through the opening 14. When the photographing device 11 and the energy generating device 12 are carried in and out, it means that the photographing device 11 and the energy generating device 12 move to be located inside the robot 10 or outside the robot 10 under the control of the control unit 22.

The photographing device 11 may have a cylindrical bar shape, and may rotate in a longitudinal direction of the photographing device 11 as shown in FIG. 7. However, as in FIG. 6, the photographing device 11 is not limited to a cylindrical bar shape. As only the photographing device 11 rotates, the photographing device 11 may scan surroundings of the robot 10. In other words, as the photographing device 11 rotates, the photographing device 11 may output a real-time image obtained by photographing the surroundings of the photographing device 11 to the display device 21. A medical expert or an operator may determine a position of a tumor cell by referring to the real-time image, and control the photographing device 11 to photograph the tumor cell.

The energy generating device 12 may have a cylindrical bar shape like the photographing device 11, may generate energy on a front surface of the energy generating device 12, and transmit the generated energy towards a tumor cell. However, as in FIG. 6, the energy generating device 12 is not limited to a cylindrical bar shape. Although the energy generating device 12 transmits energy to a side surface in FIG. 6, the energy generating device 12 may transmit energy to a front surface in FIG. 7.

Since the photographing device 11 photographs from a side surface of the robot 10, a length of the energy generating device 12 protruding through the opening 14 may be less than a length of the photographing device 11 protruding through the opening as shown in FIG. 7. The control unit 22 may set an energy transmission angle of the energy generating device to transmit energy to a region which the photographing device 11 photographs.

Since surroundings of the robot 10 may be scanned by rotating only the photographing device 11 in the robot 10 of FIG. 6 or 7, constructed as described above, whether a tumor cell exists around the robot 10 may be easily determined. Also, since the photographing device 11 and the energy generating device 12 slide through or are carried in and out through the opening 14 based on the control signal of the control unit 22, the photographing device 11 and the energy generating device 12 may be protected from damage and may operate only when needed.

Although the photographing device 11 and the energy generating device 12 have cylindrical shapes in FIGS. 6 and 7, the present embodiments are not limited thereto. Each of the photographing device 11 and the energy generating device 12 may have any of various shapes, such as, a polygonal shape, a rectangle shape, an ovular shape, and the like.

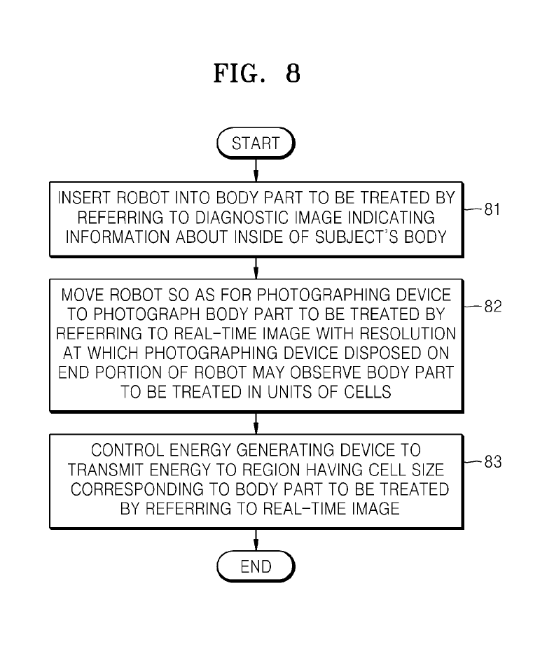

FIG. 8 is a flowchart illustrating a method of controlling the robot system 100, according to an example embodiment of the present disclosure. Referring to FIG. 8, the method includes operations which may be sequentially or selectively performed by the control device 20 of FIG. 2. Although omitted here, the description of the control device 20 given above applies to the method of FIG. 8. The method of controlling the robot system 100 performed by the control device 20 includes the following operations.

In operation 81, the control device 20 inserts the robot 10 into a region adjacent to a subject's body part to be treated by referring to a diagnostic image indicating information about the inside of the subject's body. The control device 20 receives the diagnostic image from the imaging device 30, and inserts the robot 10 into the body part to be treated by using a 3D coordinate system indicating positions of the robot 10 and the subject. The control device 20 may receive coordinates or a movement direction to or in which the robot 10 is to move, and moves the robot 10 to the coordinates or in the movement direction based on the diagnosis image.

In operation 82, the control device 20 moves the robot 10 such that the photographing device 11 may photograph the body part to be treated by referring to a real-time image with a resolution at which the photographing device 11 of the end portion 40 of the robot 10 may observe the body part to be treated in units of cells. The control device 20 moves the robot 10 so as for the photographing device 11 to more accurately photograph the body part to be treated. In other words, when the robot 10 is initially inserted into the subject's body, since the robot 10 is inserted into the region adjacent to the body part to be treated, the photographing device 11 may not accurately photograph the body part to be treated. Accordingly, the control device 20 may move the robot 10 such that the photographing device 11 may photograph the body part to be treated by referring to the real-time image received from the photographing device 11. Also, the control device 20 may rotate the photographing device 11 so as for the photographing device 11 to photograph the body part to be treated. The control device 20 may enable a medical expert or operator to refer to the real-time image by displaying the real-time image received from the photographing device 11 on the display device 21.

In operation 83, the control device 20 controls the energy generating device 12 to transmit energy to a region having a cell size corresponding to the body part to be treated by referring to the real-time image. For example, the control device 20 may set an energy transmission direction of the energy generating device 12 based on a direction of photographing of the photographing device 11. For example, the direction of energy transmission may be set to be a direction in which the photographing device 11 performs photographing. The control device 20 receives at least one of a type, an intensity, a range, and a transmission angle of energy through the control unit 22, and controls the energy generating device 12.

As described above, according to the one or more of the above embodiments of the present disclosure, since a real-time image with a resolution at which a subject's body part to be treated may be observed in units of cells is captured and energy is transmitted to the body part to be treated by referring to the real-time image, a robot system using a surgical robot and a method of controlling the robot system may precisely remove a tumor cell.

The embodiments of the present disclosure may be written as computer programs and may be implemented in general-use digital computers that execute the programs using a computer readable recording medium. Also, a data structure used in the method may be recorded by using various units on a computer-readable recording medium. The results produced can be displayed on a display of the computing hardware. A program/software implementing the embodiments may be recorded on non-transitory computer-readable media comprising computer-readable recording media. Examples of the computer-readable recording medium include magnetic storage media (e.g., ROM, floppy disks, hard disks, etc.), optical recording media (e.g., CD-ROMs, or DVDs), etc.

Further, according to an aspect of the embodiments, any combinations of the described features, functions and/or operations can be provided.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A robot system comprising a surgical robot inserted into a subject, the robot system comprising:
a photographing device disposed on the surgical robot that is inserted into a body part of the subject, which is to be treated, and captures an image;
a control device that receives a control signal for controlling the surgical robot by referring to the image received from the photographing device; and
an energy generating device that is disposed near the photographing device, and transmits energy to a region corresponding to the body part to be treated according to the control signal.

2. The robot system of claim 1, wherein the photographing device is disposed on an end portion of the surgical robot, the image is a real-time image with a resolution at which the both part to be treated may be observed in units of cells, and the energy generating device is disposed adjacent to the photographing device transmitting energy to the region which is being photographed by the photographing device.

3. The robot system of claim 1, wherein the control device moves the photographing device and the energy generating device to the body part to be treated by using a three-dimensional (3D) coordinate system that indicates positions of the surgical robot and the subject obtained by referring to a diagnostic image indicating information about the inside of the subject's body.

4. The robot system of claim 1, wherein the photographing device and the energy generating device are disposed on a side surface of an end portion of the surgical robot and are rotatable.

5. The robot system of claim 4, wherein a plurality of the photographing devices and a plurality of the energy generating devices are alternatingly arranged along a longitudinal direction of the surgical robot.

6. The robot system of claim 1, wherein the photographing device and the energy generating device slide through an opening formed in a front surface of an end portion of the surgical robot.

7. The robot system of claim 6, wherein the energy generating device transmits energy to the body part to be treated located in front of the surgical robot.

8. The robot system of claim 1, wherein the photographing device and the energy generating device are independently rotatable.

9. The robot system of claim 1, wherein the energy generating device transmits energy for activating a material for removing a tumor cell of the body part to be treated.

10. The robot system of claim 9, wherein the material is a nanomaterial or a molecular material that is attached to the tumor cell and reacts to a specific energy of the transmitted energy.

11. The robot system of claim 1, wherein the control device sets an energy transmission direction of the energy generating device to be in a direction in which the photographing device performs photographing.

12. The robot system of claim 1, further comprising an auxiliary photographing device that is disposed on a front surface of an end portion of the surgical robot and photographs a travel direction of the surgical robot.

13. The robot system of claim 1, further comprising an imaging device that obtains a diagnostic image by photographing the subject and transmits the diagnostic image to the control device,
wherein the diagnostic image is a 3D image.

14. The robot system of claim 1, wherein the control device controls the energy generating device to transmit energy according to the control signal.

15. The robot system of claim 1, wherein the surgical robot further comprises a probe for injecting medicine or a surgical tool that is disposed on an end portion of the surgical robot.

16. The robot system of claim 1, wherein the surgical robot is one of a linear robot, a flexible robot, and a multi-joint robot,
wherein the linear robot has a bar shape, the flexible robot has a softly bent shape, and the multi-joint robot includes a plurality of bars combined at joints.

17. A method of controlling a robot system including a surgical robot inserted into a subject, the method comprising:
inserting the surgical robot into a body part of the subject, which is to be treated;
receiving a control signal for controlling the surgical robot by referring to an image by using a photographing device disposed on the surgical robot; and
controlling an energy generating device that transmits energy to a region corresponding to the body part to be treated according to the control signal, by referring to the image.

18. The method of claim 17, wherein the image is a real-time image with a resolution at which the body part to be treated may be observed in units of cells, and the photographing device is disposed on an end portion of the surgical robot.

19. The method of claim 17, wherein the inserting of the surgical robot comprises inserting the surgical robot into the body part to be treated by using a three-dimensional (3D) coordinate system that indicates positions of the surgical robot and the subject obtained by referring to a diagnostic image captured by an imaging device.

20. The method of claim 18, further comprising receiving the real-time image from the photographing device and displaying the real-time image.

21. The method of claim 17, wherein the controlling of the energy generating device comprises controlling the energy generating device by receiving at least one of a type, an intensity, a range, and a transmission angle of the energy.

22. The method of claim 17, wherein moving of the surgical robot comprises rotating the photographing device, such that the photographing device photographs the body part to be treated.

23. The method of claim 17, wherein the photographing device sets an energy transmission direction of the energy generating device to be a direction in which the photographing device performs photographing.

24. A computer-readable recording medium having embodied thereon a program for executing the method of claim 17.
